(19) United States
(12) Patent Application Publication
(10) Pub. No.: US 2024/0139487 A1
(43) Pub. Date: May 2, 2024

(54) IMPLANTABLE CRANIAL MEDICAL DEVICE

(71) Applicant: Cerebral Therapeutics, Inc., Aurora, CO (US)

(72) Inventors: Dwight A. Skinner, New Brighton, MN (US); Felix E. Robles Toth, Duxbury, MA (US)

(73) Assignee: Cerebral Therapeutics, Inc., Aurora, CO (US)

(21) Appl. No.: 18/385,674

(22) Filed: Oct. 31, 2023

Related U.S. Application Data
(60) Provisional application No. 63/421,566, filed on Nov. 1, 2022.
(60) Provisional application No. 63/455,335, filed on Mar. 29, 2023.

(51) Int. Cl. A61M 39/04; A61M 1/00

(52) U.S. Cl. CPC A61M 39/04; A61M 1/77; A61M 2039/025

(57) ABSTRACT

An implantable cranial medical device includes a housing configured to be positioned on a skull of a subject such that at least a portion of the housing covers a burr hole of the skull. The medical device also includes a brain catheter connector extending from the housing and configured to extend within the skull. The brain catheter connector includes a first connector and a second connector. The second connector extends farther away from the housing than the first connector. Additionally, the medical device includes a first fluid path extending through the housing and the first connector, and a second fluid path extending through the housing and the second connector. The first and second fluid paths are not in fluid communication with one another within the housing.

### PRIORITY CLAIM

This application claims priority to U.S. Provisional Application No. 63/421,566, filed Nov. 1, 2022, and U.S. Provisional Application No. 63/455,335, filed Mar. 29, 2023, both entitled “IMPLANTABLE CRANIAL MEDICAL DEVICE,” the disclosures of which are incorporated herein by reference in their entireties.

### FIELD

The present disclosure relates to, among other things, implantable cranial medical devices, such as devices for delivering fluid to cerebrospinal fluid (CSF), such as cerebral ventricles, withdrawing or draining fluid from CSF, such as cerebral ventricles, or delivering fluid to CSF and withdrawing or draining fluid from CSF.

### INTRODUCTION

Delivery of therapeutic agents to the central nervous system (CNS) and treatment of diseases of the CNS present challenges. For example, many therapeutic agents are not able to reach the brain at therapeutic concentrations when administered through traditional routes due to the blood-brain-barrier (BBB). In addition, systemic concentrations of therapeutic agents, or metabolites or degradation products thereof, may be undesirably high to achieve therapeutic levels in the brain when therapeutic agents that do cross the BBB are systemically administered.

Some devices and therapies have been developed to administer therapeutic agents to CSF to address some of these challenges. Such devices and therapies have typically been configured to deliver a bolus of therapeutic agent to a cerebral ventricle or to chronically administer the therapeutic agent to intrathecal space of the spinal canal. Such approaches have shortcomings for treatment of diseases of the brain. For example, such approaches lack the ability to achieve adequate spatially and temporally exposure of a therapeutic agent. Bolus administration of a therapeutic agent may not provide consistent therapeutically effective concentrations of the therapeutic agent, and intrathecal administration may not provide for sufficiently high concentrations of therapeutic agent in the brain due to, for example, gravitational forces and relatively limited CSF circulation.

Monitoring of a state of a disease, subject or therapy, such as concentrations of therapeutic agents in the brain, may also present challenges. For example, accurate estimates of concentrations of therapeutic agents in the brain may not always be readily achieved through routine blood or urine analysis. In addition, withdrawing CSF to obtain more accurate estimates of central therapeutic agent concentrations is invasive and may present risks.

For those therapies that include direct intracerebral ventricular delivery of therapeutic agents through, for example, an implantable infusion pump or port, CSF may be withdrawn through a catheter configured to deliver the therapeutic agent to the CSF from the pump or port. However, withdrawing CSF through the same lumen of a catheter that has been used to deliver therapeutic agent may not provide a clean sample of CSF. That is, the CSF may include therapeutic agent that was present in the lumen of the catheter and thus may not provide for accurate estimates of concentration of the therapeutic agent in the CSF. In addition, the CSF or the components of the CSF that may be measured may interact with the material defining the lumen of the catheter, which may adversely affect subsequent delivery of the therapeutic agent. Furthermore, withdrawing CSF through the same lumen of the catheter that has been used to deliver therapeutic agent requires an interruption in the delivery of the therapeutic agent. The interruption of delivery can lead to cessation of therapy and other substantial consequences.

The use of separate catheters or separate lumens of a catheter for delivering therapeutic agents to the cerebral ventricles and for withdrawing CSF from the cerebral ventricles has been proposed. However, designing and manufacturing devices, such as access ports, that may couple to two or more catheters or a catheter having more than one lumen for delivering fluid to or from cerebral ventricles presents challenges, particularly if the devices are configured to be implanted under the scalp or skin of a subject. For example, a subject, such as a human patient, may tolerate a limited height of a device, such as a port that is coupled to the catheter or catheters, above the skull when the device is implanted under the scalp. In addition, devices implanted under the scalp or skin should have an external shape and profile configured to minimize erosion of the skin and patient discomfort and facilitate safe and efficient surgical implantation. An additional challenge relates to ensuring a secure connection between catheters and the devices to which they are connected. For example, it may be difficult to couple a catheter to an access port after the port is positioned in a subject. Further challenges relate to reproducible access to the implanted port. For example, proper positioning of the port should be maintained to ensure repeated access through the skin of a subject over extended periods of time. Such challenges result from the limited space available for such devices to be implanted, multifunctionality of the devices, and the need for secure connection between a catheter and the access port.

At least in part because of these challenges, a device of suitable size, shape, and functionality for connecting to more than one catheter or a catheter with more than one lumen for delivering fluid to or from a CSF space, such as a cerebral ventricle, of a subject has not previously been developed.

### SUMMARY

The present application relates to, among other things, an implantable cranial device that comprises two or more separate fluid flow paths. The implantable cranial device may be coupled to multi-lumen catheters, with each lumen being independently in fluid communication with one of the separate fluid flow paths. One or more of the separate fluid flow paths may be used, for example, to deliver therapeutic agents to a cerebral ventricle through one or more lumens of one or more catheters. One or more of the separate fluid flow paths may be used, for example, to withdraw CSF from a cerebral ventricle through one or more lumens of the one or more catheters.

The implantable cranial device is able to accommodate multiple separate fluid flow paths, in part, because a portion of the device is configured to be implanted within a burr hole in a skull and a portion is configured to be implanted between the skull and the scalp. The added space afforded by the portion implanted within the burr hole permits the implantable cranial device to include multiple separate fluid flow paths, while meeting design considerations to allow for safe and efficient implantation under the scalp.

According to an aspect of the present disclosure, an implantable cranial medical device may include a housing configured to be positioned on a skull of a subject such that at least a portion of the housing covers a burr hole of the skull. The implantable cranial medical device may also include a brain catheter connector extending from the housing and configured to extend within the skull. The brain catheter connector may include a first connector and a second connector. The second connector may extend farther away from the housing than the first connector. Additionally, the implantable cranial medical device may include a first fluid path extending through the housing and the first connector, and a second fluid path extending through the housing and the second connector. The first and second fluid paths may not be in fluid communication with one another within the housing.

According to another aspect of the present disclosure, a system may include an implantable cranial medical device and a brain catheter. The implantable cranial medical device may include a housing positioned on a skull of a subject such that at least a portion of the housing is within a burr hole of the skull. The implantable cranial medical device may also include a brain catheter connector extending from the housing and configured to extend within the skull. The brain catheter connector may include a first connector and a second connector. The second connector may extend farther away from the housing than the first connector. The brain catheter may be operably couplable to the brain catheter connector. The brain catheter may define a first lumen operably couplable to the first connector to define a first fluid path and a second lumen operably couplable to the second connector to define a second fluid path.

Also provided herein are methods of implanting the implantable cranial medical devices and associated catheter or catheters, as well as methods of treating, monitoring, or treating and monitoring a disease of state of a subject using the implantable cranial medical devices. For example, the method may include providing an implantable cranial medical device comprising a housing and a brain catheter connector extending from the housing. The brain catheter connector may include a first connector and a second connector. The second connector may extend farther away from the housing than the first connector. The method may also include attaching a brain catheter to the brain catheter connector to define a first fluid path and a second fluid path through the implantable cranial medical device and the brain catheter. Attaching the brain catheter to the brain catheter connector may include coupling a second lumen of the brain catheter to the second connector such that at least a portion of the second lumen overlaps with the second connector. The second lumen may define at least a portion of the second fluid path. Attaching the brain catheter to the brain catheter connector may also include coupling a first lumen of the brain catheter to the first connector after the second lumen is coupled to the second connector. The first lumen may define at least a portion of the first fluid path.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic side view of an embodiment of an implantable cranial medical device.

FIG. 2 is a schematic bottom view of an embodiment of the implantable cranial medical device depicted in FIG. 1.

FIG. 3 is a schematic top view of an embodiment of the implantable cranial medical device depicted in FIGS. 1 and 2.

FIG. 4 is a schematic perspective view of an embodiment of an implantable cranial medical device.

FIG. 5 is a schematic side view of an embodiment of an implantable cranial medical device depicted in FIG. 4.

FIG. 6 is a schematic top view of an embodiment of the implantable cranial medical device depicted in FIG. 4.

FIG. 7 is a schematic side view of the implantable cranial medical device depicted in FIG. 4 in which the housing is sectioned.

FIG. 8 is a schematic perspective view of the implantable cranial medical device depicted in FIG. 4 in which the housing is sectioned.

FIG. 9 is a schematic cross-sectional view of an embodiment of a dual lumen brain catheter.

FIG. 10 is a schematic longitudinal sectional view of a distal portion of a brain catheter.

FIG. 11 is a schematic longitudinal sectional view of a distal portion of a brain catheter.

FIG. 12 is an exploded perspective view of an embodiment of an implantable cranial medical device showing components of a port and an external catheter connector.

FIG. 13 is a perspective view of an embodiment of an implantable cranial medical device coupled to a brain catheter and a stylet employed for implanting the brain catheter while the brain catheter is coupled to the implantable cranial medical device.

FIG. 14 is a schematic sectional view illustrating an embodiment of an implantable cranial medical device, brain catheter, and external catheter implanted in a patient.

FIG. 15 is a schematic sectional view illustrating some components of an embodiment of implantable cranial device with fluid flow pathways shown in dashed lines.

FIG. 16 is a schematic drawing of a system including an implantable cranial medical device and an external device.

FIG. 17 is a schematic drawing of a system including an implantable cranial medical device and two external devices.

FIGS. 18-21 are schematic illustrations of some components of embodiments of systems described herein.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components

### DETAILED DESCRIPTION

The present disclosure relates to, among other things, an implantable cranial medical device. The implantable cranial medical device includes two catheter connectors extending from a housing of the implantable cranial medical device. One of the catheter connectors extends farther away from the housing to a distal end than a distal end of the other catheter connector. The orientation and positioning of the stepped catheter connectors (e.g., the distance of distal ends from the housing being different) may assist in attaching a catheter (e.g., a side-by-side dual lumen catheter) to the catheter connectors. Specifically, the present disclosure may relate to side-by-side dual lumen catheters as opposed to a dual lumen catheter in which a proximal portion is bifurcated.

For example, a first lumen of the catheter may be attached to a first catheter connector that extends farthest from the housing and then a second lumen of the catheter may be attached to a second catheter connector that is closes to the housing. As such, any resistance of attaching a lumen of a catheter to a connector may be divided between each side. For example, initially, only resistance from attaching a first lumen to the first catheter connector may be felt by the user (e.g., an implanting physician) and thereafter only resistance from attaching the second lumen to the second catheter connector may be felt by the user. On the other hand, if the distal ends of the catheter connectors were equal distance from the housing, the user may experience resistance from attaching both lumens on the corresponding catheter connectors. In other words, attaching a dual lumen catheter such that each lumen attaches to a catheter connector at the same time may result in twice the resistance or force as attaching the lumens of a dual lumen catheter to stepped catheter connectors (e.g., distal ends of the catheter connectors extending from the housing by different distances).

By arranging the catheter connectors in this way, the process of attaching a dual lumen catheter may use less force. Therefore, the user may be able to attach the catheter to the implantable cranial medical device by themselves more easily (and, e.g., as opposed to the catheter being attached prior to being delivered to the end user). The user may implant the catheter within the body before attaching the catheter to the medical device. For example, the user may insert a stylet or guide wire within the catheter to appropriately position the catheter and then the catheter may be attached to the medical device (e.g., as compared to using a stylet or guide wire through the medical device after the catheter is already attached).

Further, by allowing the catheter to be more easily attached to the medical device by the user, the user may trim the catheter before attaching to the medical device. The catheter may define a specific structure or features at a distal end of the catheter (e.g., an end of the catheter implanted into the body) that is opposite a proximal end of the catheter that is attached to the medical device. Therefore, the distal end of the catheter may not be trimmed to size for a specific application. However, because the user can easily attach the catheter to the medical device, the proximal end of the catheter may be trimmed (e.g., to trim the length of the catheter to size) before being attached to the medical device.

A trimmable catheter may allow the user to customize the length of the catheter being implanted. Typically catheters are manufactured in a variety of lengths, and the user selects a length suitable for the patient at hand. To be trimmable, the catheter should be sufficiently rigid at room temperature to prevent deformation. Specifically, the catheter may be sufficiently compliant at body temperature to avoid damage to tissue associated with movement of the tissue relative to the implanted catheter.

Therefore, a dual lumen catheter, having side-by-side lumens, defining a unitary outer diameter at the proximal end (e.g., as a result of manufacturing or trimming) may be manually connected to the catheter connectors. On the other hand, prior designs (e.g., not having a stepped connector as described herein) includes the manufacturer connecting the catheter to the port of the medical device prior to shipping to, e.g., ensure proper and robust connection. Because of this, prior designs could not provide a trimmable catheter (e.g., due to the special arrangement of the openings at the distal end and being unable to trim a proximal end that is already connected to the medical device). Additionally, this presented some implant process difficulties associated with the prior designs such as, e.g., a stylet would need to be inserted through the port of the medical device and into the catheter to implant the catheter into the body, rather than implanting the catheter on its own through more standard procedures and then coupling the catheter to the port of the medical device.

The present disclosure relates to an implantable cranial medical device, and systems and methods thereof, including a housing configured to be positioned on a skull of a subject (e.g., a patient) such that at least a portion of the housing covers a burr hole of the skull. The implantable cranial medical device may comprise one or more brain catheter connectors. For example, the implantable cranial medical device may include a brain catheter connector extending from the housing and configured to extend within the skull. For purposes of the present disclosure, a “brain catheter connector” is a connector operably couplable to a catheter that is configured to extend from the implantable cranial medical device into a brain of a subject, such as into a CSF-containing space (e.g., a cerebral ventricle), when implanted in the subject. Specifically, the brain catheter connector may include a first connector and a second connector. The second connector may extend farther away from the housing than the first connector.

The implantable cranial medical device may also include a first fluid path and a second fluid path. The first fluid path may extend through the housing and the first connector. The second fluid path may extend through the housing and the second connector. The first and second fluid paths may not be in fluid communication with one another within the housing. In other words, the fluid flow paths are separate and isolated in the housing.

The fluid flow paths may be formed in any suitable manner and form any suitable material or materials. The fluid flow paths, or portions thereof, may include passageways formed from one or more parts. In some examples, the passageway may be formed from a lumen within tubing. In some examples, the passageway may be formed from a channel in a solid part that is coupled to a second part to enclose the passageway. In some embodiments, the fluid flow paths may be formed by the housing. For example, the fluid flow paths may be formed in a mold of the housing, micromachined in a component of the housing, may be formed by void space generated by assembling components of the housing, or the like. In one or more embodiments, the material lining the passageway may be compatible with fluid that flows through the passageway, such as a fluid containing a therapeutic agent or CSF. In one or more embodiments, the therapeutic agent or excipients or CSF do not sorb to or react with the material defining the passageway. For purposes of the present disclosure, “sorb” refers to one or both of adsorb and absorb.

The system of the present disclosure may include the implantable cranial medical device and one or more catheters. In other words, the implantable cranial medical device may be configured to operably couple to one or more catheters. The one or more catheters may be configured for delivering fluids to, or withdrawing fluid from, a tissue or fluid of a central nervous system of a subject. In one or more embodiments, a dual lumen catheter (e.g., a side-by-side dual lumen catheter) may be configured to operably couple to the catheter, where a first lumen of the multi-lumen catheter is in fluid communication with the first fluid flow path and a second lumen of the catheter is in fluid communication with the second fluid flow path. The first fluid flow path and the first lumen may be configured to deliver fluid to, or withdraw fluid from, a first target brain location. The second fluid flow path and the second lumen may be configured to deliver fluid to, or withdraw fluid from, a second target brain location. The first and second target brain locations may be the same or different.

The brain catheter may define one or more openings in communication with a lumen of the catheter through which fluid may flow. The brain catheter may include any suitable number of openings in communication with each lumen. For example, the brain catheter may include one to ten or more openings in communication with each lumen, such as two to six openings or three to four openings in communication with each lumen. The brain catheter may have the same number of openings in communication with the first lumen as in communication with the second lumen. The brain catheter may have a different number of openings in communication with the first lumen than in communication with the second lumen.

Preferably, at least one of: (i) the first fluid flow path and first lumen; and (ii) the second fluid flow path and second lumen, is configured to withdraw CSF from a subject, for example, from a cerebral ventricle. For purposes of the present disclosure, withdrawing CSF from a CSF-containing space, such as a cerebral ventricle, includes aspirating or draining CSF from the CSF-containing space. In some embodiments, both (i) the first fluid flow path and first lumen and (ii) the second fluid flow path and second lumen, are configured to deliver fluid to, or withdraw fluid from, a CSF-containing space of a subject. In one or more embodiments, one fluid flow path and lumen are used to deliver fluid to a CSF-containing space, such as a cerebral ventricle, and the other fluid flow path and lumen are used to withdraw fluid from a CSF-containing space, such as a cerebral ventricle.

The implantable cranial device is able to accommodate multiple separate fluid flow paths, in part, because a portion of the device is configured to be implanted within a burr hole in a skull and a portion is configured to be implanted between the skull and the scalp. The added space afforded by the portion implanted within the burr hole permits the implantable cranial device to include multiple separate fluid flow paths, while meeting design considerations to allow for safe and efficient implantation under the scalp.

The brain catheter connector may be configured to be operably coupled to a proximal end of a brain catheter. In one or more embodiments, the brain catheter may define a side-by-side dual lumen at the proximal end. Therefore, the proximal ends of the lumens may be at the same longitudinal position along the catheter. In such configuration, one lumen may be attached to the connector before the other lumen, e.g., due to the stepped characteristics of the connector (e.g., distal ends of the connectors at different distances from the housing of the medical device). Further, the brain catheter may be trimmed and easily inserted onto the brain catheter connector.

In one or more embodiments, the first connector may extend from the housing by 3 to 7 millimeters (e.g., about 5 millimeters) and the second connector may extend from the housing by 4 to 8 millimeters (e.g., about 6 millimeters).

In one or more embodiments, a distal end of the first connector may be spaced apart from a distal end of the second connector by a distance of about 1 millimeter (e.g., between 0.5 millimeters and 2 millimeters) measured along a connector axis (e.g., along which the brain catheter connector extends from the housing).

In one or more embodiments, the first and second connectors may be spaced apart by about 1 millimeters (e.g., between 0.5 millimeters and about 2 millimeters) in a direction transverse to the connector axis (e.g., measured center to center of the lumens of the connectors).

Each of the brain catheter connectors may define a lumen extending therethrough. For example, the first connector may define a first lumen and the second connector may define a second lumen. The first and second lumens may extend through the respective connector to a distal opening at a distal end of the corresponding connector. Therefore, the first fluid path may extend through the housing and be in fluid communication with the distal opening of the first connector and the second fluid path may extend through the housing and be in fluid communication with the distal opening of the second connector.

A brain catheter connector of the implantable cranial medical device may be coupled to a brain catheter in any suitable manner. For example, the brain catheter and the brain catheter connector may comprise quick-release couplings, luer lock fittings, snap connect couplings, or the like. In some examples, the brain catheter may be configured to be coupled to the brain catheter connector via interference fit. The brain catheter connector may include a fitting, which may comprise external barbs, configured to be inserted into a lumen of the brain catheter to retain the brain catheter relative to the brain catheter connector and to place the lumen of the brain catheter in fluid communication with the fluid flow path of the implantable cranial medical device. For example, each of the first and second connectors may include barbs extending from an outer surface of the connector. For example, the barbs may be configured to help retain the catheter attached to the connector. Specifically, the catheter may extend over the barbs when the catheter is attached to the connector, but the barbs may provide additional resistance to removing the catheter from the connector. The connectors may include any suitable number of barbs. The barbs may be arranged and configured relative to the connectors in any suitable way. For example, the barbs may completely encircle the outer surface of the first and second connectors.

The housing may be formed from one or more parts. In some embodiments, the housing comprises a single part that defines an exterior surface, an upper flange portion and a lower portion of the housing. The housing may be formed from more than one part, the parts may be connected in any suitable manner. As an example, different parts of the housing may be secured relative to one another by threaded engagement, snap fit engagement, interference fit engagement, may be welded, adhered, or otherwise bonded to one another, or the like, or combinations thereof. For example, different parts of the housing may be connected to one another in a fluid tight manner. Also, different parts of the housing may be welded together.

The housing of the implantable cranial medical device may comprise any suitable material. For example, the housing of the implantable cranial medical device may be formed from one or more of a metallic material, a plastic material, a ceramic material, and a glass material. For example, the housing may comprise one or more of a high performance thermoplastic or relatively rigid plastic material, such as polyurethane, polycarbonate, polysulfone, polyether ether ketone (PEEK), nylon, and Ultra High Molecular Weight Polyethylene (UHMWPE); and a biocompatible metal, such as a stainless steel alloy, titanium, and nitinol. In one or more embodiments, the material may be compatible with magnetic resonance imaging (MRI). In one or more embodiments, the housing may include a biocompatible material or comprises an exterior biocompatible coating.

In one or more embodiments, the housing may include (e.g., be formed of) titanium at sections through which the first and second fluid paths extend. In other words, the portions of the medical device that interface with fluids (e.g., medications, body fluid, etc.) may be formed from titanium. Further, the housing may include (e.g., be formed of) plastic at sections not defining the first and second fluid paths. In other words, the portions of the medical device that do not interface with fluids (e.g., medications, body fluid, etc.) may be formed from plastic. By forming portions of the medical device from different materials, the medical device may achieve various benefits. For example, titanium portions of the medical device may help maintain robustness (e.g., to chemicals) while the plastic portions of the medical device may decrease the overall weight (e.g. compared to having the entire device formed of titanium), reduce the amount of metal present (e.g., to limit potential interactions with magnetic resonance imaging), and provide increased flexibility to conform to the skull.

The implantable cranial medical device may comprise any suitable connector or port to permit fluid to flow to or from a flow path of the device through a separate device, such as a catheter, needle, or the like. For example, the implantable cranial medical device may include a port defining an opening in the housing. The connector or port may be formed, at least in part, the upper flange portion or the lower portion of the device. For example, the first or second openings of the upper flange portion or the lower portion may define the connector or port, or a portion thereof. The connector or port may be, at least in part, separate from, but operably coupled to, the upper flange portion or the lower portion. For example, the connector or port may be operably coupled to the first or second openings of the upper flange portion or the lower portion to place the connector or port in communication with the fluid flow path. Specifically, the port may be in fluid communication with the first fluid path.

In one or more embodiments, the implantable cranial medical device may also include a septum. The septum may seal the opening of the port while allowing fluid communication to external fluid pathway by way of a syringe that punctures the septum. In one or more embodiments, the septum may be a self-sealing septum. A self-sealing septum may allow multiple cycles of needle insertion into and withdrawal from the port while continuing to seal the port from an interstitial environment in which the port may be located when implanted. The implantable cranial medical device may include any suitable self-sealing septum. For example, the self-sealing septum may include silicone, a polyethylene, of the like, and combinations thereof.

In one or more embodiments, the implantable cranial medical device may also include an external catheter connector in communication with the second fluid path. The external catheter connector of the implantable cranial medical device may be coupled to the catheter in any suitable manner. For example, the external catheter and the external catheter connector may comprise quick-release couplings, luer lock fittings, snap connect couplings, or the like. In some examples, the external catheter is configured to be coupled to the external catheter connector via interference fit. The external catheter connector may comprise a fitting, which may comprise external barbs, configured to be inserted into a lumen of the external catheter to retain the external catheter relative to the external catheter connector and to place the lumen of the external catheter in fluid communication with the fluid flow path of the implantable cranial medical device.

In one or more embodiments, the external catheter connector has a longitudinal axis that extends substantially parallel to a bottom of the housing. In other words, the external catheter connector may extend in a direction similar to the bottom surface of the housing. Further, the external catheter connector may extend substantially perpendicular to a direction of the connectors extending from the housing. As such, the external catheter connector may extend away from the housing along the skull of the patient in a low profile manner.

In one or more embodiments, the housing may define a plurality of fastener openings configured to receive fasteners to attach the implantable cranial medical device to the skull. The housing may define any suitable number of fastener openings to attach the device. In other embodiments, the housing may be attached to the skull in any suitable way.

In one or more embodiments, the implantable cranial medical device may also include an upper flange portion configured to rest on the skull about the burr hole and a lower portion configured to be placed within the burr hole. The lower portion may be sized and shaped to be disposed within a burr hole in a skull of a subject. Accordingly, the size and shape of the lower portion may vary depending on the size and shape of the burr hole, or the size of the burr hole may be determined by the size and shape of the lower portion. Preferably, the clearance between an exterior side surface of the lower portion and the skull is small when the lower portion is positioned in the burr hole. The smaller the clearance between the skull and the exterior side surface of the lower portion, the larger the volume of the lower portion. Larger volumes of the lower portion may facilitate incorporation of both the first and second flow paths within the device. Smaller clearances between the exterior side surface of the lower portion and the burr hole also improve stability of the implantable cranial device relative to the skull. Improved stability may be manifest in less movement of the implantable cranial device relative to the skull.

In some embodiments, the clearance between the exterior side surface of the lower portion and the skull, when the lower portion is disposed in the burr hole, is between 0 mm and 5 mm. For example, the clearance between the exterior side surface of the lower portion and the skull, when the lower portion is disposed in the burr hole, may be between 0.1 mm and 3 mm, such as between 0.2 mm and 2 mm.

In some embodiments, the lower portion has a width in a range from about 10 millimeters to about 20 millimeters. For example, the lower portion of the housing may have a width in a range from about 10 millimeters to about 14 millimeters, such as from about 11 millimeters to about 13 millimeters. A burr hole is typically cylindrical. In some embodiments, the burr hole may have a diameter of about 14 millimeters. The lower portion of the device may have a generally circular cross-section, and the width of the lower portion may be a diameter.

When the implantable cranial medical device is implanted, a bottom major surface of the lower portion preferably does not extend substantially beyond the bottom of the burr hole. Accordingly, the height of the lower portion of the housing of the implantable cranial medical device may vary depending on the thickness of the skull of the subject into which the device is implanted. As an example, a thickness of a human adult skull may typically be in a range from about 6.5 millimeters to about 7 millimeters.

In some embodiments, the height of the lower portion of the implantable cranial medical device is in a range from about 3 millimeters to about 7 millimeters. For example, the height of the lower portion may be in a range from about 4 millimeters to about 6 millimeters or from about 4.5 millimeters to about 5.5 millimeters.

The upper flange portion is preferably configured to rest on a skull of a subject above the burr hole. For purposes of this disclosure, the upper flange portion will be considered to rest on the skull if one or more intervening structures are placed between the bottom of the upper flange portion and the skull. The upper flange portion preferably has a width greater than the burr hole so that the upper flange portion may rest on the skull above the burr hole when the lower portion is positioned in the burr hole. Accordingly, the upper flange portion preferably has a width greater than the lower portion.

The upper flange portion may have a top surface and a bottom surface. The bottom surface of the upper flange portion may laterally extend relative to the lower portion. The bottom surface of the upper flange portion may be annular. The bottom surface of the upper flange portion may be generally flat. For purposes of the present disclosure, “generally flat,” in the context of the bottom surface of the upper flange portion includes slightly curved to approximate curvature of a skull. The bottom surface may be generally flat and annular.

The upper flange portion may have any suitable width. In some embodiments, the width is defined by the bottom surface of the upper flange portion. In some embodiments, the upper flange portion has a width in a range from about 15 millimeters to about 30 millimeters. For example, the upper flange portion may have a width in a range from about 18 millimeters to about 28 millimeters, or from about 22 millimeters to about 26 millimeters. The upper flange portion may have an outer surface that has a generally circular cross section. Accordingly, the width of the upper flange portion may be a diameter.

The upper flange portion may have any suitable height. In one or more embodiments, the height of the upper flange portion is sufficiently small to be well tolerated by a subject when the implantable cranial medical device is implanted under a scalp of the subject. The height of the upper flange portion may define the distance that the implantable cranial medical device extends above the skull when implanted in the subject.

In some embodiments, the height of the upper flange portion of the implantable cranial medical device is in a range from about 3 millimeters to about 8 millimeters. For example, the height of the upper flange portion may be in a range from about 4 millimeters to about 6.5 millimeters or from about 5 millimeters to about 6.5 millimeters.

The implantable cranial medical device may have any suitable overall height. In some embodiments, the height of the device from the bottom of the lower portion to the top of the upper flange portion of the housing is in a range from about 6 millimeters to about 15 millimeters, such as from about 8 millimeters to about 12 millimeters.

The top surface of the upper flange portion may have any suitable shape. In one or more embodiments, the top surface or the transition from a side surface to the top surface has no sharp edges. In some embodiments, the top surface of the housing has a generally convex shape.

In some embodiments, the bottom surface of the upper flange portion of the housing is generally flat and annular and the top surface is generally convex.

In some embodiments, the thickness or height of the upper flange portion increases moving from an outer edge of the upper flange portion towards the center the upper flange portion.

Further, the implantable cranial medical device, and systems and methods thereof, may include a variety of different components as described in U.S. patent application Ser. No. 17/856,758, entitled “IMPLANTABLE CRANIAL MEDICAL DEVICE,” which is herein incorporated by reference in its entirety.

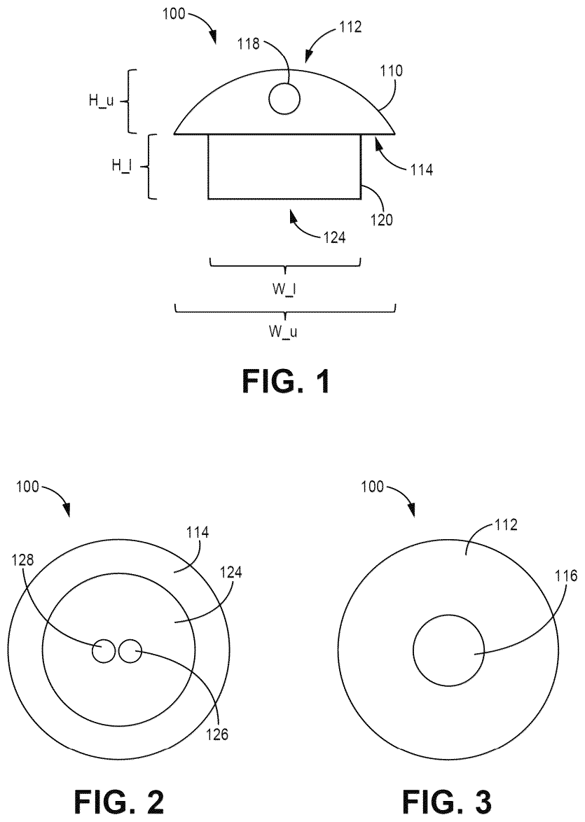

Reference is now made to FIGS. 1-3, which illustrate an example of an embodiment of an implantable cranial medical device 100 of the present disclosure. The implantable cranial medical device 100 comprises an upper flange portion 110 and a lower portion 120. The upper flange portion has a generally convex top surface 112 and a generally flat bottom surface 114. The upper flange portion 110 is configured to be positioned between a skull and a scalp of a subject when implanted, with the bottom surface 114 configured to rest on a skull of a subject. The upper flange portion 110 has a height H_u and a width W_u. The height H_u of the upper flange portion 112 is sufficiently small to avoid skin erosion and substantial discomfort to the subject when implanted. The width W_u of the upper flange portion 112 is sufficiently large to rest on the skull around a burr hole.

The lower portion 120 has a bottom surface 124, a height H_1 and a width W_1. The height H_1 of the lower portion 120 is sufficiently small so that the bottom major surface 124 of the lower portion 120 does not extend substantially below the burr hole when implanted in a subject. In one or more embodiments, the bottom major surface 124 of lower portion 120 may not extend below the burr hole when implanted in a subject. The width W_1 of the lower portion 120 may be sufficiently small such that the lower portion 120 fits within the burr hole when implanted.

The upper flange portion 110 may define a first opening 116 and a second opening 118. The lower portion 120 may define a first opening 126 and a second opening 128. A first fluid flow path (not shown in FIGS. 1-3) may extend within a housing 101 from the first opening 116 of the upper flange portion 110 to the first opening 126 of the lower portion 120. A second fluid flow path (not shown in FIGS. 1-3) may extend within the housing 101 from the second opening 118 of the upper flange portion 110 to the second opening 128 of the lower portion 120.

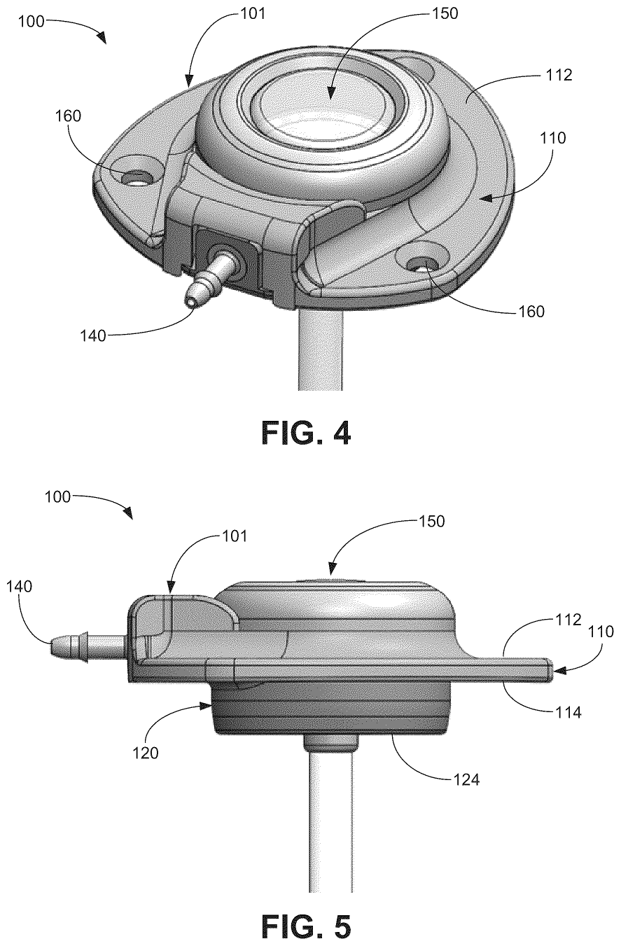

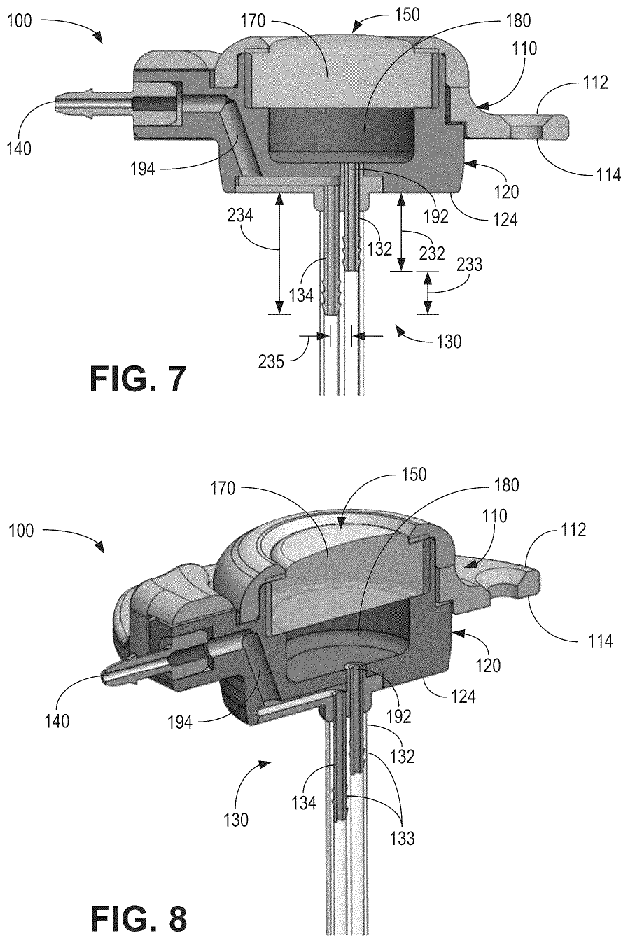

Reference is now made to FIGS. 4-8 in which an embodiment of an implantable cranial medical device 100 is shown. As with the embodiment shown in FIGS. 1-3, the embodiment of an implantable cranial medical device 100 shown in FIGS. 4-8 includes an upper flange portion 110 having a top surface 112 and a bottom surface 114 and includes a lower portion 120 having a bottom major surface 124. Fastener feedthroughs 160 extend through the upper flange portion 110 from the top surface 112 to the bottom surface 114 and are configured to receive fasteners, such as screws, which may be used to anchor the housing to the skull of a subject in which the device 100 is implanted.

The device 100 includes a port 150 in communication with the first fluid path 192. The lateral edge of the port 150 is defined by the first opening 116 defined by the top surface 112 of the upper flange portion 110. A self-sealing septum 170 extends across the opening 116. When the device 100 is implanted, the port 150 is positioned and oriented to receive a needle inserted through the scalp such that a lumen of the needle is placed in fluid communication with the first fluid path 192.

The device 100 includes a reservoir 180 in communication with and forming a part of the first fluid path 192. The reservoir 180 is positioned such that insertion of a needle into the port 150 places a lumen of the needle in fluid communication with the reservoir 180. The reservoir 180 is disposed with the housing 101 and may form a part of the first fluid flow path 192.

The device also includes an external catheter connector 140 that extends substantially parallel with the bottom surface 114 of the upper flange portion 110 of the device 100. The external catheter connector 140 is operably coupled to an opening defined by the upper flange portion 110 (such as second opening 118 depicted in FIG. 1). The external catheter connector 140 defines a passageway in communication with a second fluid path 194 of the device 100. The first fluid path 192 and the second fluid path 194 are not in fluid communication with one another within the device 100. The external catheter connector 140 is configured to operably couple to an external catheter to place a lumen of the external catheter in fluid communication with the second fluid path 194. The external catheter may further be coupled to a device other than the implantable cranial infusion device, such as an infusion device. The infusion device may be implantable or non-implantable.

The implantable cranial medical device 100 also includes a brain catheter connector 130 having a first connector 132 (e.g., defining a first lumen) and a second connector 134 (e.g., defining a second lumen). The brain catheter connector 130 extends from the bottom major surface 124 of the lower portion of the housing 101. The first connector 132 of the brain catheter connector 130 is operably coupled to the first fluid path 192 (e.g., the first connector 132 defining a first lumen therethrough). The second connector 134 of the brain catheter connector 130 is operably coupled to the second fluid path 194 (e.g., the second connector 134 defining a second lumen therethrough). The first and second connectors 132, 134 may define a lumen extending therethrough to a distal opening at a distal end of the respective connector.

The second connector 134 extends farther away from the implantable cranial medical device 100 (e.g., the bottom major surface 124 of the lower portion 120) than the first connector 132 (e.g., the first and second connectors 132, 134 extend from the bottom major surface 124 along a connector axis). For example, the second connector 134 extends from the bottom major surface 124 by a distance 234 (e.g., measured along the connector axis) of about 6 millimeters (e.g., between about 4 to 8 millimeters) and the first connector 132 extends from the bottom major surface 124 by a distance 232 (e.g., measured along the connector axis) of about 5 millimeters (e.g., between about 3 to 7 millimeters). Further, a distal end of each of the first and second connectors 132, 134 are spaced apart by a distance 233 (e.g., measured along a length of the connectors 132, 134 (e.g., the connector axis)) of about 1 millimeter (e.g., between about 0.5 to 2 millimeters). Further yet, the first and second connectors 132, 134 are spaced apart (e.g., between a center of a lumen of each of the first and second connectors 132, 134) by a distance 235 (e.g., measured transverse to a length of the connectors 132, 134 (e.g., transverse to the connector axis)) of about 1 millimeter (e.g., between about 0.5 to 2 millimeters).

The brain catheter connector 130 is configured to operably couple to a dual lumen catheter, which may have a distal end. The distal end may be implantable in a CSF-containing space, such as a cerebral ventricle.

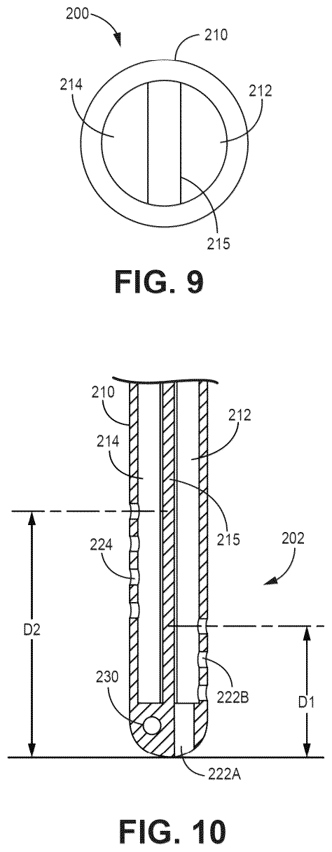

For example and with reference to FIG. 9, a central portion 215 of a body 210 of a brain catheter 200 that separates a first lumen 212 from a second lumen 214 may be inserted between the brain catheter connectors 130 such that the first lumen 212 of the catheter 200 is placed in fluid communication with the first connector 132 of the brain catheter connector 130 and such that the second lumen 214 of the catheter 200 is placed in fluid communication with the second connector 134 of the brain catheter connector 130. Additionally, in one or more embodiments, the brain catheter may define a side-by-side dual lumen catheter.

Each of the connectors 132, 134 include barbs 133 located proximate the distal end of the connector 132, 134. The barbs 133 are configured to be inserted into a lumen of the corresponding catheter. Due to the offset positioning of the distal ends of the connectors 132, 134, the catheter is inserted over the barbs 133 of the second connector 134 before being inserted over the barbs 133 of the first connector 132. The barbs 133 help to restrict movement of the catheter from being removed from the connectors 130.

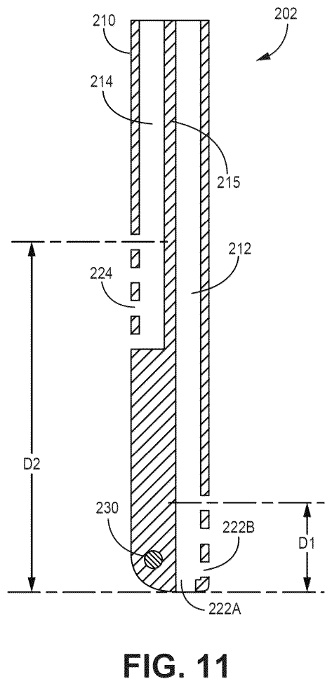

Referring now to FIGS. 10 and 11, distal portions 202 of embodiments of brain catheters are shown. The brain catheter includes a first lumen 212 and a second lumen 214 separated by a central wall 215 of the body 210 of the catheter. The first lumen 212 is in communication with one or more openings 222 at the distal end portion 202. In the depicted embodiment, the first lumen 212 is in communication with three side openings 222B at the distal end portion 202 and a distal end opening 222A. The second lumen 214 is in communication with one or more side openings 224 at the distal end portion 202. In the depicted embodiment, the second lumen 212 is in communication with four side openings 224 at the distal end portion 202. The side openings 222A and 224 are positioned on generally opposing sides of the catheter. The distance (D1) from the distal tip to the side opening 222A furthest from the distal tip is less than the distance (D2) from the distal tip to the side opening 224 furthest from the distal tip. Preferably, all the openings 224 in communication with the second lumen 214 are positioned away from the distal tip a distance greater than D1. The position and orientation of the openings 222A, 222B, 224 allow for therapeutic fluid that may be infused through the second lumen through side openings 224 to substantially mix with CSF prior to being aspirated through openings 222A, 222B and through the first lumen 212.

In the embodiment depicted in FIG. 11, D2 is substantially greater than D1. Accordingly, the catheter depicted in FIG. 11 may be suitable for delivering fluid to, or withdrawing fluid from, for example, a cerebral ventricle through openings 222A and 222B and may be suitable for delivering fluid to brain parenchyma through openings 224.

In the embodiment depicted in FIG. 10, the difference between D2 and D1 is not as larger as depicted in FIG. 11. Accordingly, the catheter depicted in FIG. 10 may be suitable for delivering fluid to, or withdrawing fluid from, for example, a cerebral ventricle through openings 222A and 222B and through openings 224.

In the embodiment depicted in FIGS. 10 and 11, the catheter includes a radiopaque marker 230 visible by X-ray, magnetic resonance imaging (MM), or X-ray and MM imaging so that the location of the distal end portion 202 may be readily determined. The depicted radiopaque marker 230 comprises tantalum and is embedded in the distal end of the catheter. The body 210 may comprise barium sulfate or another radiopaque material (not shown) dispersed throughout. In some embodiments (not shown), the brain catheter may include radiopaque markers at locations along the length of the catheter to form depth markings that may be used to determine the depth of the catheter during or after implantation.

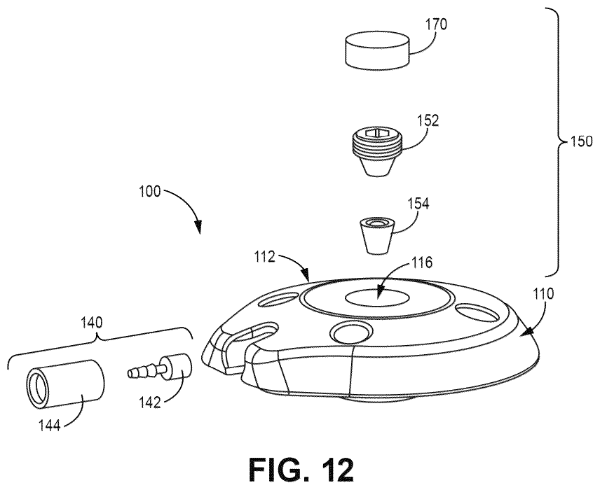

Referring now to FIG. 12, components of an external catheter connector 140 and a port 150 associated with an upper flange portion 110 of an implantable cranial medical device 100 are shown. The outer edge of the port 150 is defined by a first opening 116 defined by a top surface 112 of the upper flange portion 110. The port 150 includes a septum 170 or cap, a compression wedge 152, and an axial ferrule 154, which defines a decreasing inner diameter moving in a direction from the top surface 112 of the upper flange portion 110 towards the bottom surface of the upper flange portion 110. The axial ferrule 154 serves to guide a needle inserted into the port 150 into a position suitable for placing a lumen of the needle in fluid communication with the first fluid path of the device 100. The axial ferrule 154 may also limit insertion of the needle beyond a bottom end of the ferrule 154.

The external catheter connector 140 includes a compression fitting, such as sleeve 144, defining a lumen configured to receive an external catheter and a barbed fitting 142 configured to be inserted into a lumen of the external catheter. The barbed fitting 142 includes a passageway in communication with the second fluid path of the device 100. The compression sleeve may be disposed about the external catheter and slid towards the end of the catheter after the catheter has been advanced over the barbed fitting 142 to cause the external catheter to be compressed against the barbed fitting 142.

The implantable cranial medical device described herein may be included in a kit. The kit may include a brain catheter. The brain catheter may be coupled to the implantable cranial medical device in the kit or may be coupled by an end user, such as a healthcare professional. As described herein, the brain catheter may be coupled to the implantable cranial medical device by the end user such that the end user may trim the catheter prior to attachment and may position the catheter within the body prior to attachment. The kit may include a compression fitting to aid in connecting an external catheter to an external catheter connector of the implantable cranial medical device. The kit may include fasteners, such as screws, configured to secure the upper flange portion of the implantable cranial medical device to the skull of a subject. The kit may include a tool, such as a screwdriver, for use with the fasteners. The kit may include a needle configured to access a port of the implantable cranial medical device, which may be used for, for example, aspiration. The needle may be a non-coring needle, such as a Huber needle or a butterfly needle.

While only a few kit components are listed above, it will be understood that any component discussed herein may be included in a kit comprising an implantable cranial medical device.

The implantable cranial medical devices and associated devices described herein may be implanted in any suitable manner. In some embodiments, a brain catheter having a distal end and a proximal end is implanted such that the distal end is positioned in a CFS-containing region, such as a cerebral ventricle, of a subject. The proximal end may be positioned in proximity to a burr hole in a skull. In one or more embodiments, the brain catheter is a side-by-side dual lumen catheter. The proximal end of the catheter may be coupled to the implantable cranial infusion device to place one lumen of the brain catheter in communication with the first fluid path of the implantable cranial medical device and the second lumen of the brain catheter in communication with the second fluid path of the implantable cranial medical device.

Specifically, a second lumen of the brain catheter may be coupled to the second connector such that at least a portion of the second lumen overlaps with the second connector and a first lumen of the brain catheter may be coupled to the first connector after the second lumen is coupled to the second connector. The first lumen may define at least a portion of the first fluid path and the second lumen may define at least a portion of the second fluid path.

In one or more embodiments, attaching the brain catheter to the brain catheter connector may include rotating the brain catheter after the second lumen is coupled to the second connector and before the first lumen is coupled to the first connector. For example, the brain catheter may be rotated to assist in inserting the second lumen of the brain catheter over the second connector. Thereafter, the first lumen of the brain catheter may be inserted over the first connector. In other words, the brain catheter may be coupled to the first and second connectors in a sequential manner (e.g., the second lumen is inserted over the second connector and then the first lumen is inserted over the first connector).

In one or more embodiments, the first and second lumens of the brain catheter may be side-by-side at a proximal end of the brain catheter (e.g., the first and second lumens of the brain catheter may be attached or connected at the proximal end). The brain catheter may be couplable to the brain catheter connector at the proximal end.

In one or more embodiments, the user may trim the brain catheter to a desired catheter length prior to attaching the brain catheter to the brain catheter connector.

In one or more embodiments, trimming a brain catheter may include trimming a proximal end of the brain catheter and attaching a brain catheter to the brain catheter connector may include attaching the proximal end of the brain catheter to the brain catheter connector. For example, because the user may attach the brain catheter to the connector (e.g., not attached during manufacturing), the proximal end of the brain catheter may be trimmed to a desired length prior to attaching. A distal end of the brain catheter may maintain an initial shape for use with the implantable cranial medical device while customizing the overall length of the brain catheter by trimming the proximal end.

In one or more embodiments, the user may couple an infusion device to the second fluid path of the implantable cranial medical device and may infuse fluid from the infusion device to the second fluid path.

In one or more embodiments, attaching the brain catheter to the brain catheter connector may include extending the first and second lumens over barbs extending from an outer surface of the first and second connectors, respectively. The barbs may include any suitable structure or component to assist with retaining the brain catheter on the respective connector. Further, the barbs may be oriented (e.g., angled towards the housing) such that the lumens of the brain catheter may be inserted over the barbs with relative ease as compared to removing the lumens from the barbs (e.g., restricting or maintaining the position of the brain catheter).

In one or more embodiments, the user may implant a distal end of the brain catheter in a cerebrospinal fluid (CSF)-containing space of a subject (e.g., patient).

In one or more embodiments, the CSF-containing space may be a cerebral ventricle

In one or more embodiments, the user may implant the implantable cranial medical device on a skull of a subject such that at least a portion of the housing is covering a burr hole of the skull. For example, the implantable cranial infusion device may be implanted such that the lower portion is disposed in the burr hole and the upper flange portion is between a skull and a scalp of a subject.

In one or more embodiments, implanting the implantable cranial medical device on the skull may include coupling the implantable cranial medical device to the skull via fasteners through a plurality of fastener holes defined by the housing. For example, the upper flange portion may be secured to the skull via one or more fasteners, such as screws.

In one or more embodiments, the user may place a lumen of a needle in communication with the first fluid path of the implantable cranial medical device and aspirating the fluid from a CSF-containing space of the subject through the lumen of the needle.

In one or more embodiments, the user may infuse a fluid to a CSF-containing space of a subject by infusing the fluid through the second flow path of the implantable cranial medical device and through the second lumen of the brain catheter.

The brain catheter may be coupled to the implantable infusion device before or after the distal end of the brain catheter is positioned in the cerebral ventricle. In one or more embodiments, the brain catheter may be coupled to the implantable cranial medical device after the brain catheter is implanted. A stylet may be inserted through the brain catheter to facilitate implanting the catheter into the brain of a subject.

A subject, in which the implantable cranial medical device and associated components may be implanted, may undergo a preoperative scan for surgical planning. A surgical navigation system may be employed during an implant procedure to facilitate placement of the brain catheter. The brain catheter may be radiopaque, may include one or more radiopaque markers, or may be radiopaque and include one or more radiopaque markers to allow visualization with the surgical navigation system employed. For example, Medtronic, Inc.'s AxiEM™ electromagnetic technology and StealthStation® Navigation System and B. Braun Aesculap division's Intraventricular Disposable Introducer Set™ may be used to facilitate implantation of the catheter. Preferably, the distal end portion of the catheter is positioned in a cerebral ventricle of the subject. Preferably, the distal end portion of the catheter is positioned in a lateral ventricle. However, the distal end portion of the brain catheter may be placed in any suitable location, such in the cisterna magna, a subarachnoid space, or the like.

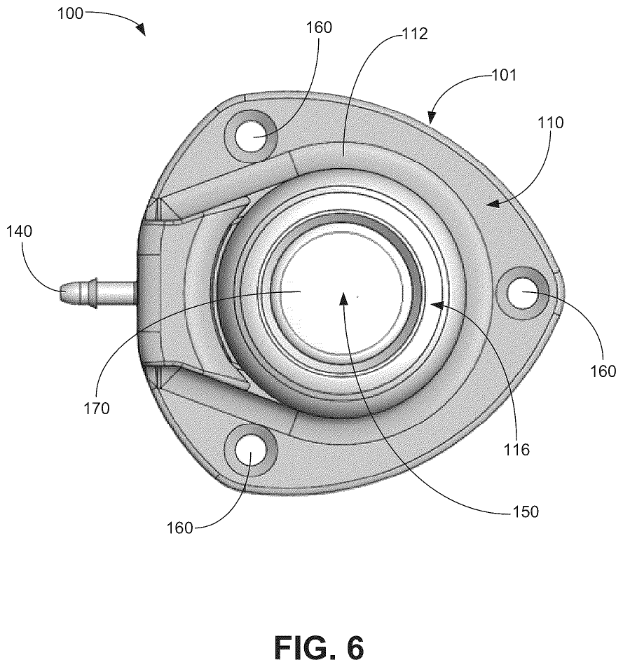

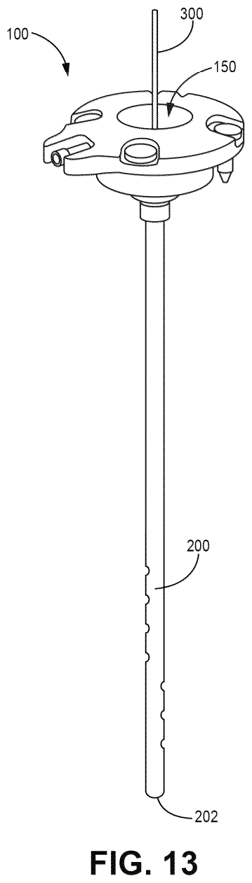

Referring now to FIG. 13, an example illustrating an implantable cranial medical device 100 and coupled brain catheter 200 is shown. The device 100 and brain catheter 200 are shown prior to the catheter 200 being implanted. As shown in FIG. 13, a stylet 300 may be inserted into the port 150 of the implantable cranial medical device 100, through the first fluid path (such as fluid path 192 shown in FIG. 6) and at least partially through a first lumen (such as lumen 212 shown in FIG. 9) to facilitate implanting the distal end 202 of the catheter 200 in a cerebral ventricle of a subject. The brain catheter 200 is typically flexible and lacks rigidity. The stylet 300 may provide structural rigidity to allow insertion of the distal end 202 of the catheter 200 through brain tissue and into the cerebral ventricle. As noted herein, the brain catheter 200 may be implanted using the stylet 300 prior to attachment to the implantable cranial medical device 100.

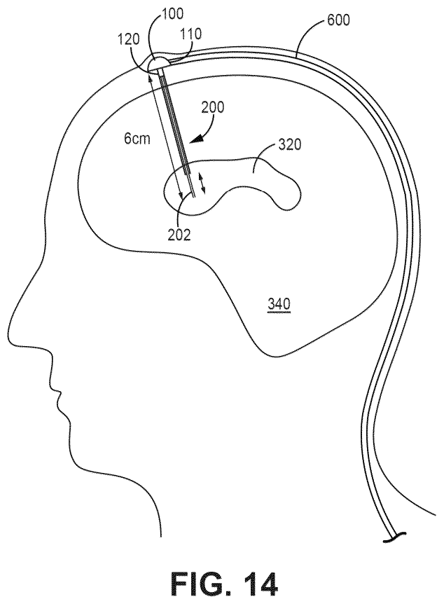

Further, and as shown in FIG. 14, the distal end portion 202 of the brain catheter 200 may be implanted in a lateral ventricle 320 of a brain 340 of a subject. The proximal end of the brain catheter 200 may be coupled to a brain catheter connector (not shown) of the device 100. The lower portion 120 of the implantable cranial medical device 100 may be positioned within a burr hole (not shown) of a skull (not shown) of the subject, and the upper flange portion 110 of the device 100 may be implanted between the skull and the scalp of the subject. An external catheter 600 may be coupled to an external catheter connector (not shown) of the implantable cranial medical device 100 and positioned along the skull of the subjected and tunneled to a position in the patient remote from the implantable cranial medical device 100. For example, the external catheter 600 may be operably coupled to an infusion device implanted subcutaneously in a location of a torso, such as in the abdomen region, of the subject.

The brain catheter 200 may have any suitable length, such as about 6 centimeters to about 7 centimeters, which should be sufficient to connect to the implantable cranial medical device 100 and extend into the lateral ventricle 320 of the brain 340 of the subject. Further, as described herein, the brain catheter 200 may be trimmed (e.g., at the proximal end) to the desired length prior to being attached to the implantable cranial medical device 100.

The implantable cranial medical devices and associated devices described herein may be used in any suitable manner. If the distal end portion of the brain catheter is positioned in a CSF-containing space of the subject, the implantable cranial medical device may be permit infusion of therapeutic fluid and aspiration of CSF through separate lumens of the brain catheter and through separate fluid paths of the device.

For example, fluid may be aspirated from the CSF-containing space of the subject through the first lumen of the brain catheter and through the first fluid path of the implantable cranial medical device. A lumen of a needle may be placed in communication with the first fluid path of the implantable cranial medical device. For example, the needle may be inserted into a port in communication with the first fluid path. The fluid may be aspirated from the CSF-containing space of the subject through lumen of the needle.

In some embodiments, the first fluid path of the implantable cranial medical device and the first lumen of the brain catheter may be used to infuse fluid, such as a fluid comprising a therapeutic agent, into the CSF-containing space of the subject. For example, a lumen of a needle may be placed in communication with the first fluid path of the implantable cranial medical device. For example, the needle may be inserted into a port in communication with the first fluid path. The fluid may be infused through the lumen of the needle, through the first fluid path of the implantable cranial medical device, and through the first lumen of the brain catheter to the CSF-containing space of the subject.

A fluid may be infused to the CSF-containing space of the subject by infusing the fluid through the second flow path of the implantable cranial medical device and through the second lumen of the brain catheter. The fluid may comprise a therapeutic agent. An infusion device may be coupled to the second fluid path of the implantable cranial medical device, such as via an external catheter, and the fluid may be infused from the infusion device to the CSF-containing space. The infusion device may be an implanted infusion device.

By providing separate lumens and fluid paths, infusion and aspiration (or infusion) may be conducted simultaneously. Accordingly, infusion of a first therapeutic fluid does not need to be disrupted to aspirate a sample of CSF or to introduce a second therapeutic fluid to the CSF. Such lack of disruption of infusion of the first therapeutic fluid may provide for improved therapy.

The devices, kits, and systems described herein may extend the space and time a therapeutic agent is available to its brain target improving pharmacokinetics and pharmacodynamics of the therapeutic agent in the brain relative to prior approaches for direct infusion into the central nervous system.

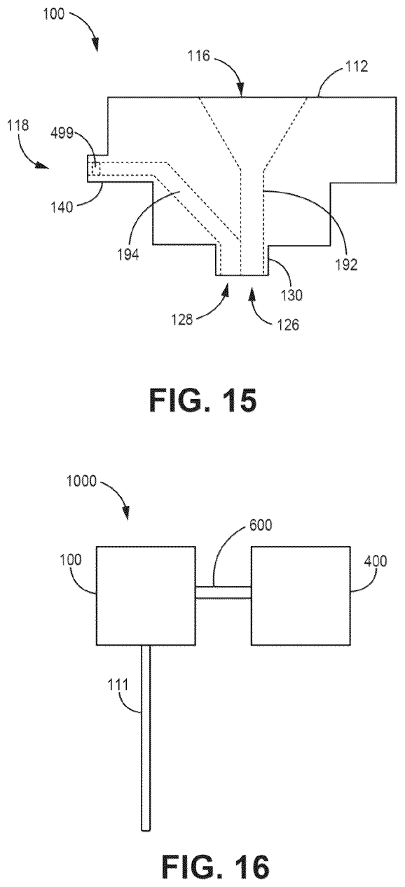

Referring now to FIG. 15, an implantable cranial device 100 includes a first fluid flow path 192 extending from a first opening 116 in the top surface 112 of the device 100 to a first opening 126 at the bottom of the device 100. The implantable cranial device 100 includes a second fluid flow path 194 extending from a second opening 118 in an upper flange portion of the device 100 to a second opening 128 at the bottom of the device 100. The second upper flange portion opening 118 in the depicted embodiment is defined by an external catheter connector 140. The first 126 and second 128 openings of the bottom of the device are formed by a brain catheter connector 130. The filter 499 is depicted as being in the second pathway 194 in proximity to the external catheter connector 140. However, the filter 499 may be positioned at any suitable location in the second pathway 194.

The implantable cranial medical devices described herein and associated devices, such as brain catheters, external catheters, and the like may be used with any suitable external device or external devices. The external devices may be infusion devices. The infusion device may be implantable or non-implantable. The non-implantable infusion device may be an ambulatory device or a stationary device. The infusion device may be manually powered, electromechanically powered, chemically powered, or otherwise powered. In some examples, the infusion device may comprise a piston pump, a peristaltic pump, an osmotic pump, a plunger, or the like. A distal portion of the CSF shunt or drainage catheter may be placed in any suitable location of the patient, such as the peritoneal cavity.

In some embodiments, more than one external device is connected to the implantable cranial medical device in serial. For example, the devices may be “daisy-chained.” In some examples, a passive infusion device is fluidly positioned between the implantable cranial medical device and an active infusion device, such a powered infusion device.

The passive device may be a modified Ommaya or Rickman reservoir. The reservoirs may be modified such that they do not need to be coupled to a catheter implanted in the brain. Rather the reservoirs may be operably coupled to the implantable cranial medical device, such as through the second fluid path, to deliver therapeutic fluid to the brain via the second lumen of the brain catheter. The reservoirs may be implanted under the scalp. The reservoirs may contain ports for introducing needles percutaneously to deliver therapeutic fluids. The reservoirs may comprise appropriate valves to ensure one-way direction of flow from the reservoir to the implantable cranial medical device.

The use of such reservoirs may be beneficial if multiple manual infusions of therapeutic agent are anticipated. Rather than repeatedly percutaneously accessing the port of the implantable cranial medical device to both aspirate CSF and infuse therapeutic fluid, the reservoir may be used introduce the therapeutic fluid, thereby reducing the number of times the skin in punctured over the port of the implantable cranial medical device.

The passive infusion device or port may be fully implantable, partially implantable or positioned external to a subject.

The port may be placed at a location to allow convenient access for introduction of therapeutic fluid into the CSF-containing space of the subject. For example, the second port may be located in proximity to the subject's ear (“auricular”) or in proximity to the subject's clavicle (“clavicular”). The port may be configured to be implanted subcutaneously on the skull in proximity to an ear or in implanted subcutaneously in proximity to a clavicle.

The port may be used to introduce a fluid to a CSF-containing space via the implantable cranial medical device. The fluid may be a therapeutic fluid. The port may be used to deliver the therapeutic fluid until the therapy is determined to be effective. For example, the port may be used to determine an appropriate therapeutic agent for use in the therapeutic fluid, an appropriate dose range, or the like. Once the therapy is determined effective, an infusion device may be operatively coupled to the implantable cranial medical device to deliver the therapeutic fluid to the brain, e.g., to a CSF-containing space. The port may be replaced with the infusion device. Alternatively, the port and the infusion device may both be operatively coupled with the implantable cranial medical device.

Some therapies involve periodic, rather than continuous, infusion of therapeutic fluid. For such therapies, the port may be used to chronically deliver periodic infusions of the therapeutic fluid to a CSF-containing space via the implantable cranial medical device.

Some therapies involve infusion of more than one therapeutic fluid. For such therapies, an infusion device operatively coupled to the implantable cranial medical device may be used to deliver a first therapeutic fluid to a CSF-containing space via the implantable cranial medical device, and the port may be used deliver a second a second therapeutic fluid to the CSF-containing space via the implantable cranial medical device. The port may be used to deliver auxiliary therapeutic fluids, to treat symptoms that may arise during infusion of the first therapeutic fluid, or the like.

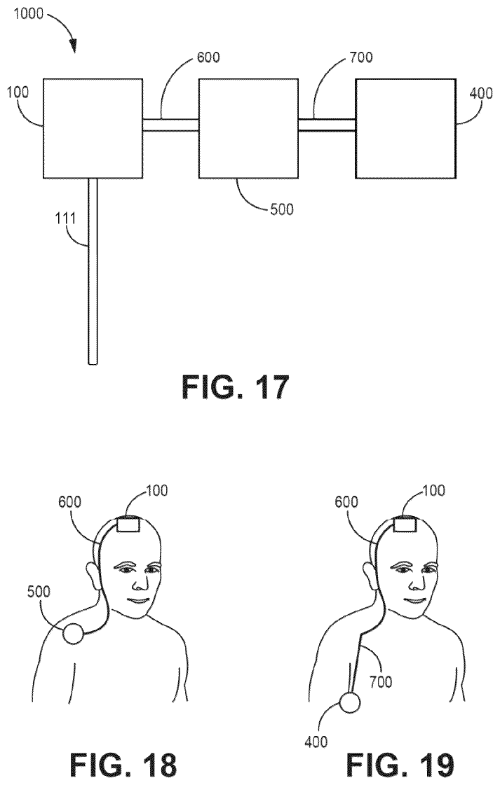

Referring now to FIGS. 16 and 17, schematic drawings of systems 1000 are shown. The systems include an implantable cranial medical device 100 and associated brain catheter 111. The system 1000 also includes an external catheter 600 operably coupled to a first 400 or second 500 external device. In FIG. 16, the implantable cranial medical device 100 is connected to the first device 400 via the external catheter 600. In FIG. 17, the implantable cranial medical device 100 is connected to the second device 500 via the external catheter 600, and the second device 500 is connected to the first device 400 via an additional catheter 700. The first device 400 may be an implantable infusion device, such as a powered implantable infusion device. The second device 500 may be a reservoir having a port accessible through the scalp of a subject, such as a modified Ommaya or Rickman reservoir.

Reference is now made to FIGS. 18-21, which illustrate some components of embodiments of systems described herein. FIG. 18 shows an implantable cranial medical device 100 implanted in a subject's head and a port 500 in proximity to the subject's clavicle. The port 500 may be implanted or may be external to the subject. The implantable cranial medical device 100 and port 500 may be implanted in, or positioned in proximity to, any other suitable location of the subject. A catheter 600 operatively couples the implantable cranial medical device 100 to the port 500.

In FIG. 19, the port 500 is disconnected from the catheter 600, and an infusion device catheter 700 that is operatively coupled to an infusion device 400 is coupled to catheter 600. The infusion device catheter 700 may be coupled to catheter 600 in any suitable manner. For example, a splice connector (not shown) having components similar to those described above regarding catheter connectors may be employed to couple the infusion device catheter 700 to catheter 600. Preferably, the infusion device 400 is implanted in the subject.

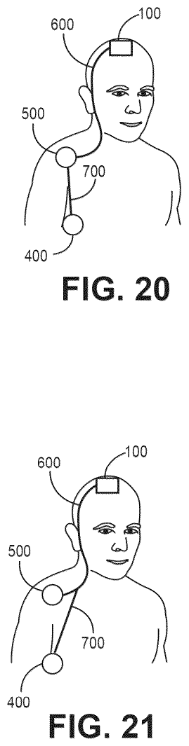

FIG. 20 illustrates an embodiment where an infusion device 400 is operatively coupled to the port 500 via an infusion device catheter 700. The infusion device catheter 700 may be coupled to an additional inlet (not shown), such as via a catheter connector (not shown), of the port 500. Preferably, the port 500 and the infusion device 400 are implanted.

FIG. 21 illustrates an embodiment in which an infusion device 400 is operatively coupled to catheter 600 via an infusion device catheter 700 while the port 500 is also coupled to catheter 600. Catheter 600 may be bifurcated at its proximal end portion to permit both the port 500 and the infusion device 400 to be simultaneously operatively coupled to the catheter 600. Alternatively, a Y-adapter or T-adapter (not shown) having components such as those described above regarding catheter connectors may be employed to operatively couple both the port 500 and the infusion device 400, via the infusion device catheter 700, to catheter 600.

The devices, systems, kits and methods described above are primarily described in the context of their use for delivering therapeutic fluids directly to the brain or withdrawing CSF from a region of the brain. It will be understood that the implantable cranial medical devices, catheters, systems, kits, and methods described herein may be used to deliver therapeutic agents to, or withdraw CSF from, the intrathecal space.

The devices, systems and devices, systems, kits and methods may be modified, as appropriate to deliver fluid to the intrathecal space. For example, the implantable cranial medical device may be modified for implanting in fascia rather than under the scalp of a subject. The device may be implanted in any suitable location of the patient, such as in the torso of the subject, near the back of the neck of the subject, or the like.

The shape of the device may be modified to accommodate the site of implantation. While the device may not maintain the shape, size and dimensions of the implantable cranial medical device described above, the functionality of the device may remain largely unchanged. That is, the device may comprise a first and second fluid flow paths and connector for connecting one or more catheters (referred hereinafter as a “spinal catheter”), preferably a multi-lumen spinal catheter, that may be placed in communication with the intrathecal space. The device may comprise a port into which needles may be inserted to deliver therapeutic fluid to, or withdraw CSF from, the intrathecal space via the first fluid flow path and first lumen of a spinal catheter. The device may comprise an external catheter connector for connecting to an external catheter to place a lumen of the external catheter in communication with the second fluid flow path. The external catheter may be used to connect to an external device or for draining CSF.

The length of a spinal catheter may be greater than the length of a brain catheter described above. The spinal catheter may be positioned in any suitable location of the intrathecal space. Preferably, the distal tip of the spinal catheter is positioned at cervical vertebral level or higher. In some embodiments, the distal tip of the spinal catheter is advanced through the intrathecal space into the cisterna magna.

The spinal catheter may have a length sufficient to be inserted into the intrathecal space and to be advanced to a higher vertebral level or the cisterna magna. The spinal catheter may be configured to enter the intrathecal space at any suitable vertebral level. For example, the spinal catheter may be configured to be introduced into the intrathecal space at a lumbar vertebral level.

The spinal catheter may have an outer diameter greater than the outer diameter of a brain catheter described above.

The spinal catheter may have multiple openings in fluid communication with an aspiration fluid flow path of the device accessible via the port. The multiple openings may be positioned any suitable distance apart, such as about three inches to about seven inches, to allow for sampling of CSF at different locations along the spine, as different biomarkers or concentrations may be present at different CSF locations. The spinal catheter may have any suitable number of openings, such as three, four, five, or more.

In some embodiments, the port has multiple aspiration fluid flow paths with each aspiration path separately coupled with a lumen of a catheter or catheters. If the catheter is a multi-lumen catheter the distal end portion of the lumens may be separated by about three inches to about seven inches along the length of the catheter so that CSF may be sampled along the length of the spinal canal. The multi-lumen spinal catheter may have any suitable number of aspiration lumens, such as three, four, five or more.

In some embodiments, the device may have multiple spinal catheter connectors with each spinal catheter connector configured to connect to a separate spinal catheter. In some embodiments, the first fluid path (the path in communication with the port) is in communication with more than one catheter port. Spinal catheters having different lengths may be coupled to the spinal catheter connectors, so that the catheters may be positioned within the intrathecal space at different vertebral levels for aspirating CSF along the length of the spinal canal.

The materials described above for the implantable cranial medical device, brain catheter, and other system and kit components may be the same for the device and spinal catheter for delivering therapeutic fluid to, or withdrawing fluid from, the intrathecal space.

The methods discussed above regarding the use of the implantable cranial medical device, systems and kits may be employed using a device, system, or kit adapted for delivering therapeutic fluid to, or withdrawing fluid from, the intrathecal space.

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

Any direction referred to herein, such as “top,” “bottom,” “side,” “upper,” “lower,” and other directions or orientations are described herein for clarity and brevity but are not intended to be limiting of an actual device or system. Devices and systems described herein may be used in a number of directions and orientations.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must).

The words “include,” “including,” and “includes” indicate open-ended relationships and therefore mean including, but not limited to. Similarly, the words “have,” “having,” and “has” also indicated open-ended relationships, and thus mean having, but not limited to. Similarly, the terms “comprise” and “comprising” indicate open-ended relationships, and thus mean comprising, but not limited to. The terms “consisting essentially of” and “consisting of” are subsumed within the term “comprising.” For example, a catheter comprising tubing may be a catheter consisting of tubing. The term “consisting essentially of” means a recited list of one or more items belonging to an article, kit, system, or method and other non-listed items that do not materially affect the properties of the article, kit, system, or method.

The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a catheter connector may be configured to place a lumen of a catheter in fluid communication with a fluid path, even when the catheter is not connected to the catheter connector).

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

What is claimed:

1. An implantable cranial medical device comprising:
a housing configured to be positioned on a skull of a subject such that at least a portion of the housing covers a burr hole of the skull;
a brain catheter connector extending from the housing and configured to extend within the skull, wherein the brain catheter connector comprises a first connector and a second connector, wherein the second connector extends farther away from the housing than the first connector;
a first fluid path extending through the housing and the first connector; and
a second fluid path extending through the housing and the second connector, wherein the first and second fluid paths are not in fluid communication with one another within the housing.

2. The implantable cranial medical device of claim 1, wherein the housing comprises titanium at sections through which the first and second fluid paths extend, and wherein the housing comprises plastic at sections not defining the first and second fluid paths.

3. The implantable cranial medical device of claim 1, further comprising a port defining an opening in the housing, wherein the port is in fluid communication with the first fluid path.

4. The implantable cranial medical device of claim 3, further comprising a septum, wherein the septum seals the opening of the port while allowing fluid communication to external fluid pathway by way of a syringe that punctures the septum.

5. The implantable cranial medical device of claim 1, further comprising an external catheter connector in communication with the second fluid path, the external catheter connector has a longitudinal axis that extends substantially parallel to a bottom of the housing.

6. The implantable cranial medical device of claim 1, wherein the housing defines a plurality of fastener openings configured to receive fasteners to attach the implantable cranial medical device to the skull.

7. The implantable cranial medical device of claim 1, further comprising:
an upper flange portion configured to rest on the skull about the burr hole; and
a lower portion configured to be placed within the burr hole.

8. A method comprising:
providing an implantable cranial medical device comprising a housing and a brain catheter connector extending from the housing, wherein the brain catheter connector comprises a first connector and a second connector, wherein the second connector extends farther away from the housing than the first connector; and
attaching a brain catheter to the brain catheter connector to define a first fluid path and a second fluid path through the implantable cranial medical device and the brain catheter, wherein attaching the brain catheter to the brain catheter connector comprises:
coupling a second lumen of the brain catheter to the second connector such that at least a portion of the second lumen overlaps with the second connector, wherein the second lumen defines at least a portion of the second fluid path, and
coupling a first lumen of the brain catheter to the first connector after the second lumen is coupled to the second connector, wherein the first lumen defines at least a portion of the first fluid path.

9. The method of claim 8, wherein attaching the brain catheter to the brain catheter connector further comprises rotating the brain catheter after the second lumen is coupled to the second connector and before the first lumen is coupled to the first connector.

10. The method of claim 8, further comprising coupling an infusion device to the second fluid path of the implantable cranial medical device and infusing fluid from the infusion device to the second fluid path.

11. The method of claim 8, further comprising implanting a distal end of the brain catheter in a cerebrospinal fluid (CSF)-containing space of a subject.

12. The method of claim 11, wherein the CSF-containing space is a cerebral ventricle.

13. The method claim 8, further comprising implanting the implantable cranial medical device on a skull of a subject such that at least a portion of the housing is covering a burr hole of the skull.

14. The method of claim 8, further comprising placing a lumen of a needle in communication with the first fluid path of the implantable cranial medical device and aspirating the fluid from a CSF-containing space of the subject through the lumen of the needle.

15. The method of claim 8, further comprising infusing a fluid to a CSF-containing space of a subject by infusing the fluid through the second fluid path of the implantable cranial medical device and through the second lumen of the brain catheter.

16. A system comprising:
an implantable cranial medical device comprising a housing positioned on a skull of a subject such that at least a portion of the housing is within a burr hole of the skull, the implantable cranial medical device further comprising a brain catheter connector extending from the housing and configured to extend within the skull, wherein the brain catheter connector comprises a first connector and a second connector, wherein the second connector extends farther away from the housing than the first connector; and
a brain catheter operably couplable to the brain catheter connector, wherein the brain catheter defines a first lumen operably couplable to the first connector to define a first fluid path and a second lumen operably couplable to the second connector to define a second fluid path.

17. The system of claim 16, wherein the first and second lumens of the brain catheter are side-by-side at a proximal end of the brain catheter, wherein the brain catheter is couplable to the brain catheter connector at the proximal end.

18. The system of claim 16, wherein each of the first and second connectors comprises barbs extending from an outer surface.

19. The system of claim 16, wherein the implantable cranial medical device further comprises a port defining an opening in the housing, wherein the port is in fluid communication with the first fluid path.

20. The system of claim 16, wherein the implantable cranial medical device further comprises:
an upper flange portion configured to rest on the skull about the burr hole; and
a lower portion configured to be placed within the burr hole.
